(19) United States
(12) Patent Application Publication
(10) Pub. No.: US 2026/0103434 A1
(43) Pub. Date: Apr. 16, 2026

(54) METHODS OF USING HYDROGEN TO EXTEND CATALYST LIFE FOR ETHANOL TO BUTADIENE CONVERSIONS

(71) Applicants: Battelle Memorial Institute, Richland, WA (US); Bridgestone Americas Tire Operations, LLC, Nashville, TN (US)

(72) Inventors: Vanessa Dagle, Richland, WA (US); Robert A. Dagle, Richland, WA (US); Terrence E. Hogan, New Franklin, OH (US)

(73) Assignees: Battelle Memorial Institute, Richland, WA (US); Bridgestone Americas Tire Operations, LLC, Nashville, TN (US)

(21) Appl. No.: 19/356,644

(22) Filed: Oct. 13, 2025

Related U.S. Application Data
(60) Provisional application No. 63/706,909, filed on Oct. 14, 2024.

(51) Int. Cl. C07C 45/29; B01J 21/06; B01J 21/08; B01J 23/50; B01J 23/86; B01J 38/10; C07C 1/207

(52) U.S. Cl. CPC C07C 45/29; B01J 21/066; B01J 21/08; B01J 23/50; B01J 23/868; B01J 38/10; C07C 1/2076; C07C 2521/06; C07C 2523/50; C07C 2523/86

(57) ABSTRACT

Disclosed herein is a method for converting ethanol to 1,3-butadiene, wherein the method utilizes H₂ produced during a first step of the method for subsequent conversions involved in the method. In particular aspects, H₂ produced from converting ethanol to acetaldehyde is used to promote the conversion of the acetaldehyde to 1,3-butadiene. The H₂ promotes enhanced catalyst stability, as well as enhanced selectivity and yields of the 1,3-butadiene product. In particular aspects, the method comprises two steps to produce the 1,3-butadiene from the ethanol and H₂ produced from a first step facilitates enhanced selectivities and yields for the second step.

### CROSS-REFERENCE TO RELATED APPLICATION

This application claims the benefit of and priority to the earlier filing date of U.S. Provisional Patent Application 63/706,909, filed on Oct. 14, 2024, the entirety of which is incorporated herein by reference in its entirety.

### ACKNOWLEDGMENT OF GOVERNMENT SUPPORT

This invention was made with government support under DE-AC0576RL01830 awarded by the U.S. Department of Energy. The government has certain rights in the invention.

### FIELD

The present disclosure is directed to a two-step method for converting ethanol to butadiene wherein H₂ is generated in a first step and used to extend catalyst life in a second step.

### PARTIES TO JOINT RESEARCH AGREEMENT

The claimed invention arose under a CRADA agreement between (i) Battelle Memorial Institute, Operator of Pacific Northwest National Laboratory, and (ii) Bridgestone Research, LLC, which agreement was in effect on or before the effective filing date of the claimed invention.

### BACKGROUND

1,3-Butadiene (BD) is an important building block used in the polymer chemistry. It is used among other things in the production of polymers such as synthetic rubbers or elastomers including styrene-butadiene rubber (SBR), polybutadiene rubber (PBR) and nitrile rubber (NR).

Currently, BD is typically primarily obtained as a by-product of the naphtha steam cracking process that serves to make ethylene. The production of ethylene from steam cracking is in decline due to the recent and drastic increase of shale gas production in the USA which offers another pathway for obtaining ethylene from ethane but results in less BD creation. In addition to the shift in lighter feedstocks, crude oil price swings have historically led to corresponding price fluctuations in the cost of butadiene, and this is not sustainable for the end users of this important building block. Thus, alternative technologies for producing butadiene are highly desired.

Ethanol conversion to butadiene (ETB) represents an attractive alternative technology. Ethanol can be sourced from a variety of locations including commercial production from renewable biomass or waste sources. Since ethanol is predominantly used as a fuel component for internal combustion engines, electric vehicle adoption will potentially lead to excess ethanol at competitive prices available for producing a wide range of commodity chemicals. Furthermore, a bioethanol pathway could be a promising alternative to the naphtha-based processes.

Achieving high yield to butadiene at industrially relevant process conditions has been challenging. There exists a need in the art for a new method that facilitates the ability to selectively produce butadiene from ethanol under efficient conditions that minimize catalyst deactivation.

### SUMMARY

Disclosed herein is a method for producing 1,3-butadiene from ethanol, the method comprising: exposing an ethanol-containing feedstock to a first catalyst system to produce a reaction product comprising acetaldehyde and H₂; and exposing the acetaldehyde of the reaction product to a second catalyst system to produce a 1,3-butadiene-containing composition, wherein exposing the acetaldehyde to the second catalyst system is done in the presence of at least a portion of the H₂ from the reaction product.

In some particular aspects, the method comprises: exposing an ethanol-containing feedstock to a first catalyst system in a first reactor to produce a reaction product comprising acetaldehyde and H₂; passing the acetaldehyde of the reaction product to a second reactor; passing at least a portion of H₂ of the reaction product to the second reactor; and exposing the acetaldehyde of the reaction product to a second catalyst system in the second reactor to produce a 1,3-butadiene-containing composition, wherein exposing the acetaldehyde to the second catalyst is done in the presence of the H₂ from the reaction product.

The foregoing and other objects, features, and advantages of the present disclosure will become more apparent from the following detailed description, which proceeds with reference to the accompanying figures.

### BRIEF DESCRIPTION OF THE DRAWINGS

FIG. 1 is a schematic illustrating a representative set-up used for the method according to aspects of the present disclosure.

FIG. 2 is a graph summarizing conversion and acetaldehyde selectivity values for converting ethanol to acetaldehyde using H₂- or N₂-rich environments.

FIG. 3 is a graph summarizing conversion and acetaldehyde selectivity values for converting ethanol to acetaldehyde at different reaction temperatures.

FIG. 4 is a graph summarizing conversion and product selectivity values for converting ethanol to acetaldehyde using a 2% Cu/SBA-16 catalyst at weight hour space velocity (WHSV) values of 6.6 hr⁻¹ and 3.0 hr⁻¹.

FIG. 5 is a graph summarizing conversion and product selectivity values for converting ethanol to acetaldehyde using a 0.2% Cr/2% Cu/SiO₂ catalyst at weight hour space velocity (WHSV) values of 0.9 hr⁻¹ and or 1.66 hr⁻¹.

FIG. 6 is a graph summarizing conversion and acetaldehyde selectivity values for converting ethanol to acetaldehyde using a 2% Cu/SBA-16 catalyst and a 2% Cu/SiO₂ catalyst.

FIG. 7 is a graph summarizing conversion and acetaldehyde selectivity values for converting ethanol to acetaldehyde using a chromium promoter to provide a 0.2% Cr/2% Cu/SBA-16 catalyst and a 0.2% Cr/2% Cu/SiO₂ catalyst.

FIG. 8 is a graph summarizing the effect on conversion values for converting ethanol to acetaldehyde using first catalyst systems comprising a chromium promoter as compared to first catalyst systems without the promoter.

FIGS. 9A and 9B are TEM images showing particle sizes of a 2% Cu/SBA-16 catalyst (FIG. 9A) and a 0.2% Cr/2% Cu/SBA-16 catalyst (FIG. 9B).

FIGS. 10A and 10B are a particle size distribution graph (FIG. 10A) and a combined XRD spectrum (FIG. 10B) illustrating the effects of using a promoter.

FIG. 11 provides graphs summarizing conversion and selectivity values towards butadiene, olefins, aromatics, and dienes for converting an ethanol and acetaldehyde mixture (2:1 by weight) to 1,3-butadiene using H₂- or N₂-rich environments and a second catalyst system comprising 4% Ag/4% ZrO2/SBA-16.

FIG. 12 provides graphs summarizing conversion and selectivity values towards butadiene, olefins, aromatics, and dienes for converting an ethanol and acetaldehyde mixture to 1,3-butadiene using H₂- or N₂-rich environments and a second catalyst system comprising 1% Ag/4% ZrO2/SBA-16.

FIG. 13 provides graphs summarizing conversion and selectivity values towards butadiene, olefins, aromatics, and dienes for converting an ethanol and acetaldehyde mixture to 1,3-butadiene using H₂- or N₂-rich environments and a second catalyst system comprising 4% ZrO2/SBA-16.

FIG. 14 is a graph summarizing conversion and 1,3-butadiene selectivity values for converting an ethanol and acetaldehyde mixture to 1,3-butadiene at different reaction temperatures.

FIG. 15 is a graph summarizing conversion values for converting an ethanol and acetaldehyde mixture (2:1 by weight) to 1,3-butadiene using second catalyst systems comprising different amounts of ZrO₂.

FIG. 16 is a graph summarizing selectivity values toward butadiene and aromatics, cyclics, and dienes for converting an ethanol and acetaldehyde mixture (2:1 by weight) to 1,3-butadiene using second catalyst systems comprising different amounts of ZrO₂.

FIG. 17 is a graph summarizing conversion and product selectivity values for converting an ethanol and acetaldehyde mixture (2:1 by weight) to 1,3-butadiene using different WHSV values.

FIG. 18 is a graph summarizing conversion and product selectivity values for converting an ethanol and acetaldehyde mixture (2:1 by weight) to 1,3-butadiene using different pressures at a WHSV of 1.8 hr⁻¹.

FIG. 19 is a graph summarizing conversion product selectivity values for converting an ethanol and acetaldehyde mixture (2:1 by weight) to 1,3-butadiene using different pressures at a WHSV of 0.9 hr⁻¹.

FIG. 20 is a graph summarizing products selectivity values for converting an ethanol and acetaldehyde mixture (2:1 by weight) to 1,3-butadiene using different H₂ partial pressures at a WHSV of 1.8 hr⁻¹ and using different amounts of an Ag dopant for the second catalyst system.

FIG. 21 is a graph summarizing conversion values for converting an ethanol and acetaldehyde mixture (2:1 by weight) to 1,3-butadiene using different H₂ partial pressures at a WHSV of 1.8 hr⁻¹ and using different amounts of an Ag dopant for the second catalyst system.

FIG. 22 is a graph summarizing conversion values for converting an ethanol and acetaldehyde mixture (2:1 by weight) to 1,3-butadiene using varying ratios of ethanol to acetaldehyde in the feedstock composition at 100 psig (7 atmospheres).

FIG. 23 is a graph summarizing conversion values for converting an ethanol and acetaldehyde mixture (2:1 by weight) to 1,3-butadiene using varying ratios of ethanol to acetaldehyde in the feedstock composition at 50 psig (3.5 atmospheres).

### DETAILED DESCRIPTION

### Overview of Terms

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

Unless otherwise indicated, all numbers expressing quantities of components, molecular weights, molarities, voltages, capacities, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing aspects of the present disclosure from discussed prior art, the stated numbers are not approximates unless the word “about” is recited.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise. Unless otherwise stated, any of the groups defined below can be substituted or unsubstituted.

In order to facilitate review of the various aspects of the disclosure, the following explanations of specific terms are provided:

Ethanol-Containing Feedstock: A composition that comprises ethanol and typically comprises at least 30% ethanol. Ethanol-containing feedstocks can be obtained from biomass, waste sources, or commercial sources.

External (or Extraneous) H₂ Source: A source of H₂ that is separate and distinct from any step or reactor (or zone) used in a method to convert ethanol to 1,3-butadiene. H₂ produced by converting an ethanol-containing feedstock to acetaldehyde using the method disclosed herein is distinct from H₂ provided by any external or extraneous H₂ source.

Promoter: A species that can be added to a first catalyst system as described herein to facilitate improved metal dispersion within the acidic support component of the first catalyst system. In some aspects, the promoter is a metal.

### INTRODUCTION

Although processes for producing butadiene exist in the art, these methods have their own drawbacks, particularly catalyst instability. Catalysts typically used in these processes are unstable under inert conditions and often decompose, even under inert conditions. Some in the art have attempted to control catalyst stability by introducing an external source of H₂ into conversion processes in the art; however, butadiene yields and catalyst viability still remain low.

The present inventors have determined that using a two-step method for converting ethanol to butadiene can facilitate enhanced butadiene yields and selectivity in view of the ability to use H₂ that is produced in the first step of the method to promote catalyst activity during the second step. In yet some additional aspects, H₂ produced in the first step can be recycled back for use in the first step and to help promote catalyst activity in the first step as well. The disclosed method can thereby avoid (or at least reduce) the need for using excess H₂ from external/extraneous sources. By extending catalyst vitality using H₂ generated from the method, reaction conditions not suitable under conventional methods can be used in the disclosed method, which can further contribute to improved conversion and selectivity values for the 1,3-butadiene obtained from the ethanol-containing feedstock.

### Method

Disclosed herein is a two-step method for making 1,3-butadiene from ethanol-containing feedstocks that utilizes conditions that simultaneously extend catalyst life of the catalyst used in the method and that improves conversion and selectivity values. In particular aspects of the disclosure, the first step of the method comprises converting an ethanol-containing feedstock to a reaction product comprising acetaldehyde and H₂. The first step typically is performed in the presence of a first catalyst system. In the second step of the method, acetaldehyde present in the reaction product is converted to 1,3-butadiene using a second catalyst system. The second step is performed in the presence of H₂ generated from the first step. In some aspects, ethanol and/or unreacted acetaldehyde (recycled from the second step) can also be used in the second step to promote formation of the 1,3-butadiene and/or conversion yields. In some aspects, any H₂ used in the second step of the method is provided entirely from the first step and is not provided by an external H₂ source (e.g., H₂ from a separate H₂ tank or the like). In some other aspects, any H₂ used in the second step of the method is provided at least partially from the first step, with other H₂ being provided by an external source. In yet additional aspects, H₂ produced from the first step can be recycled back to the first step to help stabilize the first catalyst system used to convert the ethanol of the ethanol-containing feedstock to acetaldehyde.

In some particular aspects of the present disclosure, the method comprises exposing an ethanol-containing feedstock to a first catalyst system to produce a reaction product comprising acetaldehyde and H₂, and exposing the acetaldehyde of the reaction product to a second catalyst system to produce a 1,3-butadiene-containing composition, wherein exposing the acetaldehyde to the second catalyst is done in the presence of the H₂ from the reaction product.

The first catalyst system comprises a metal and a support. In some aspects, the metal is copper, silver, or a combination thereof. In some aspects, the support is an acidic support. The amount of the metal used for the first catalyst system can range from 0.4 wt % to 20 wt %, such as 1 wt % to 15 wt %, or 1 wt % to 10 wt %, or 1 wt % to 5 wt %. In particular aspects, the metal is used at 2 wt %.

The support of the first catalyst system can be selected from any material that contains silicon and oxygen. In some aspects, the support of the first catalyst system is an acidic support, such as a silica-based material. In some aspects, the silica-based material is a mesoporous silica, a silica gel, or the like, including any combinations thereof. The silica-based material can be selected to have a surface area ranging from 100 m²/g to 500 m²/g, such as 400 m²/g to 500 m²/g. In particular aspects, the silica-based material has a surface area of 300 m²/g, 400 m²/g, 450 m²/g, or 500 m²/g. In particular aspects, the mesoporous silica is SBA-15 or SBA-16 and the silica gel is a DAVICAT® material available from W.R. Grace & Co. (e.g., DAVICAT® 57).

In some aspects, the first catalyst system can further comprise a promoter to facilitate metal dispersion on/within the support. The promoter can be selected from any Group I metal, such as sodium, potassium, or any combination thereof; any Row 4 metal, such as calcium, scandium, titanium, vanadium, chromium, manganese, iron, cobalt, nickel, copper, zinc, gallium, germanium, arsenic, selenium, or any combination thereof. In some aspects, the promoter is selected from sodium, potassium, chromium, or zinc. In some aspects, the amount of promoter can be selected to provide a ratio of copper to promoter that ranges from 10:1 to 10:5 Cu:Promoter, such as 10:1 to 10:3 Cu:Promoter. In particular examples, chromium was used as the promoter in an amount of 0.2 wt %. As described in examples herein, both 2% Cu/SBA-16 and 0.2% Cr/2% Cu/SBA-16 have small particles with an average particle size of 2% Cu/SBA-16=1.9 nm and 0.2% Cr/2% Cu/SBA-16=1.4 nm. And, particle size distribution analysis established that the majority of the particles are between 1-1.5 nm for both catalysts. The catalyst promoted with Cr shows more particles below 1 nm, which confirms that addition of a promoter can lead to smaller Cu particles and thus improve dispersion.

In particular aspects, the first catalyst system comprises copper and a silica-based material. In representative aspects, the first catalyst system comprises copper and SiO₂ gel (e.g., DAVICAT® 57), or copper and SBA-16. In some such aspects, the first catalyst system further comprises chromium. In particular representative aspects, the first catalyst system comprises 0.2 wt % Cr, 2 wt % Cu, and a SiO₂ gel support; 2 wt % Cu on an SBA-16 support; or 0.2 wt % Cr, 2 wt % Cu, and an SBA-16 support. In exemplary aspects, the first catalyst system comprised 0.2 wt % Cr, 2 wt % Cu, and an SBA-16 support.

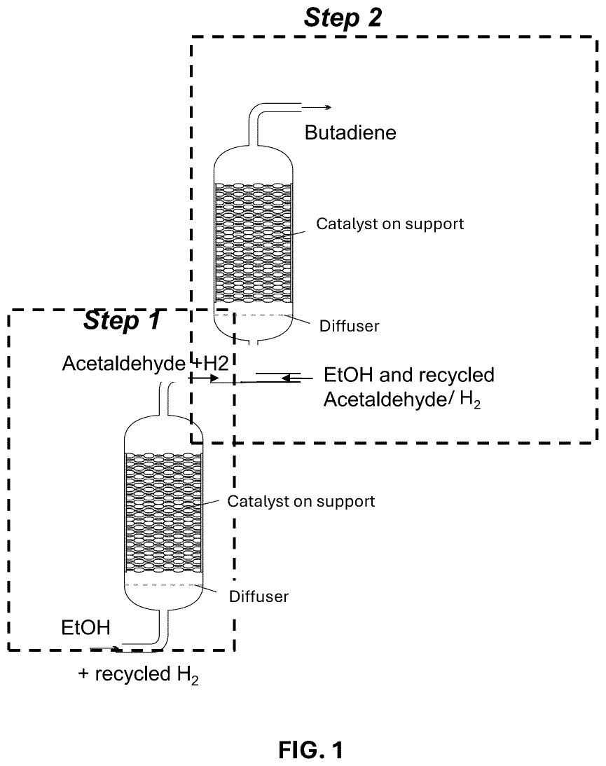

In some aspects of the disclosed method, an ethanol-containing feedstock is exposed to the first catalyst system to produce acetaldehyde in a reactor. In some aspects, this step takes place in a first reactor that is separate from a reactor used to convert the acetaldehyde to 1,3-butadiene in the second step of the method. In such aspects, the two reactors can be fluidly coupled such that an output stream produced from the first step can be passed from the first reactor and introduced as an input stream into the second reactor for the second step. Such aspects can involve using a set-up as illustrated in FIG. 1. In yet other aspects, both steps can take place in a single reactor that includes two different zones wherein the two steps can separately take place. Any suitable reactor can be used for the method, including, but not limited to, fixed bed reactors, fluidized bed reactors, packed bed reactors, tubular reactors, batch reactors, trickle-bed reactors, loop reactors, and the like. The reactors can have any suitable number of inlet/outlets capable of permitting fluid flow between the reactors and/or any external feeds (e.g., ethanol-containing feedstock feeds).

In particular aspects, the conversion of ethanol in the ethanol-containing feedstock to acetaldehyde is carried out at a temperature ranging from 300° C. to 450° C., such as 325° C. to 425° C., or 350° C. to 425° C., or 375° C. to 425° C., or 375° C. to 400° C. and at a pressure ranging from 1 psig to 200 psig, such as 1 psig to 100 psig, or 50 psig to 100 psig. In some examples, the conversion of ethanol to acetaldehyde was carried out at 400° C. and a pressure of 100 psig. A weight hour space velocity (WHSV) ranging from 0.5 hr⁻¹ to 7 hr⁻¹ can be used, such as 0.9 hr⁻¹ to 6.6 hr⁻¹, or 1 hr⁻¹ to 4 hr⁻¹, or 1 hr⁻¹ to 3 hr⁻¹, or 1 hr⁻¹ to 2 hr⁻¹. In some examples, a WHSV of 0.9 hr⁻¹, 1.66 hr⁻¹, 3 hr⁻¹, or 6.6 hr⁻¹ was used.

Exposing the ethanol-containing feedstock to the first catalyst system is typically performed under an atmosphere of H₂. In particular aspects, using an H₂-rich atmosphere facilitates higher conversion yields of ethanol to acetaldehyde, particularly when compared with performing this step under inert gases like N₂. Additionally, performing the first step of the method under an atmosphere of H₂ facilitates being able to use higher reaction temperatures, which can improve conversion and selectivity to acetaldehyde and reduce the amount of undesired byproducts, such as ethylene, diethyl ether, ethyl acetate, butyraldehyde, and the like. In some aspects, the partial pressure of H₂ used in the first step can range from 40% H₂ to 100% H₂, such as 50% H₂ to 100% H₂, or 60% H₂ to 100% H₂, or 70% H₂ to 100% H₂, or 80% H₂ to 100% H₂, or 90% H₂ to 100% H₂. In some examples, the partial pressure of H₂ was 67%, 80%, or 89%.

In some aspects, the method can comprise isolating H₂ from the reaction product produced in the first step. The isolated H₂ can be passed directly to a second reactor or second zone where acetaldehyde from the reaction product is converted to the 1,3-butadiene. In some additional aspects, the H₂ generated in the first step of the method is passed to the second reactor or second zone where acetaldehyde from the reaction product is converted to the 1,3-butadiene without isolation. In some additional aspects, a portion of the isolated H₂ produced from the first step of the method can be recycled back to the reactor (or zone) where ethanol is converted to acetaldehyde to facilitate stabilizing the first catalyst system, as discussed herein. In yet additional aspects, the method can further comprise isolating the H₂ from the 1,3-butadiene-containing composition. In some additional aspects, a portion of the H₂ from the 1,3-butadiene-containing composition is used in combination with the first catalyst system.

In some aspects of the disclosure, the first step of the method can result in conversions of ethanol to acetaldehyde ranging from 20% to 50%, such as 25% to 50%, or 30% to 50%, or 35% to 50%, or 40% to 50%, or 45% to 50% based on the amount of ethanol in the ethanol-containing feedstock. In some aspects, the first step of the method can result in selectivities of acetaldehyde ranging from 80% to 100%, such as 85% to 100%, or 90% to 100%, or 95% to 100%.

As described herein, aspects of the disclosure concern a second step of the method, which comprises exposing the acetaldehyde of the reaction product to a second catalyst system to produce a 1,3-butadiene-containing composition, wherein exposing the acetaldehyde to the second catalyst is done in the presence of the H₂ from the reaction product. In some aspects, the second step comprises using a second catalyst system, which is described below.

The second catalyst system comprises a zirconium oxide, a second catalyst support, and a metal dopant. In particular aspects, the zirconium oxide is ZrO₂. The zirconium oxide can be used in amounts ranging from 2 wt % to 12 wt %, such as 4 wt % to 8 wt %, or 4 wt % to 6 wt %, including 4 wt %, 5 wt %, or 6 wt %. In particular aspects, the zirconium oxide is used at 6 wt %. In particular aspects, the ZrO₂ is used in amounts ranging from 2 wt % to 12 wt %. In particular aspects, the second catalyst support is an acidic support.

The second catalyst support is a silica-based material in some aspects. In some aspects, the silica-based material is a mesoporous silica. The silica-based material can be selected to have a surface area ranging from 100 m²/g to 700 m²/g, such as 425 m²/g to 500 m²/g, or 450 m²/g to 500 m²/g. In particular aspects, the silica-based material has a surface area of 450 m²/g or 500 m²/g. In particular aspects, the mesoporous silica is SBA-15 or SBA-16. In representative aspects, SBA-16 is used.

The metal dopant can be selected from Ag and Cu, or a combination thereof. The metal dopant can be used in an amount ranging from 0.5 wt % to 8 wt %, such as 1 wt % to 4 wt %, or 1 wt %, 2 wt %, 3 wt %, or 4 wt %. In particular aspects, the metal dopant is Ag and is used in an amount ranging from 0.5 wt % 8 wt % or 1 wt % to 4 wt %. In particular aspects, the metal dopant is Ag and is used at 1 wt %.

In representative aspects, the second catalyst system comprises 1% Ag/4% ZrO₂/SBA-16, 1% Ag/6% ZrO₂/SBA-16, or 4% Ag/4% ZrO₂/SBA-16. In particular examples, the second catalyst system was 1% Ag/6% ZrO₂/SBA-16.

In the disclosed method, acetaldehyde from the reaction product of the first step of the method is exposed to the second catalyst system to produce a 1,3-butadiene-containing composition in a reactor. In some aspects, this second step takes place in a second reactor that is separate from the first reactor used to convert ethanol of the ethanol-containing feedstock to the acetaldehyde (e.g., see FIG. 1). Alternatively, as discussed herein, the second step can be carried out in the same reactor as the first step but in a separate zone from the first zone where the first step is carried out.

In particular aspects, the 1,3-butadiene-containing composition produced in the second step of the method can further comprise unreacted acetaldehyde. This unreacted acetaldehyde can be recycled back for use in the second step. For example, the unreacted acetaldehyde can be combined with acetaldehyde from the reaction product in the second step of the method (e.g., in the second reactor or second zone). In some aspects, the unreacted acetaldehyde can be combined with the acetaldehyde from the reaction product before or after passing the acetaldehyde from the reaction product to the second reactor or second zone. In yet other aspects, the unreacted acetaldehyde can be combined with the acetaldehyde from the reaction product at the same time as passing it to the second reactor or second zone.

In particular aspects, the unreacted acetaldehyde and/or the acetaldehyde from the reaction product are combined with the second catalyst system. In some aspects, the unreacted acetaldehyde, the acetaldehyde of the reaction product, and ethanol are combined with the second catalyst system.

In some aspects, the second step further comprises adding ethanol to the unreacted acetaldehyde and/or the acetaldehyde of the reaction product. In some aspects, the ethanol is added in an amount that provides a ratio of ethanol:total acetaldehyde ranging from 1:1 to 3:1, such as 2:1 to 3:1, or 1:1 to 2.5:1, or 2:1 to 2.5:1, wherein the total acetaldehyde represents the total amount of any unreacted acetaldehyde plus acetaldehyde from the reaction product. In representative aspects, the ethanol:total acetaldehyde ratio was 2:1.

As discussed herein, the second step of the method is conducted in the presence of H₂. This H₂ typically is provided partially or, more commonly, entirely from the H₂ generated in the first step of the method. In some aspects, the amount of H₂ used in the second step is equivalent to the amount produced in the first step. In some aspects, any H₂ used in the second step of the method consists of the H₂ produced in the first step of the method. In yet additional aspects, the H₂ used in the first step can partially comprise H₂ that has been recycled from the first step, wherein a portion of the H₂ of the reaction product from the first step is passed to the second step and wherein another portion of the H₂ of the reaction product is passed back to the first step. In some aspects, the partial pressure of H₂ that is used in the second step can be modified to control conversion rates and/or selectivity for 1,3-butadiene production, such as by reducing the amount of side-products that are produced in the second step (e.g., aromatics, cyclics, dienes other than 1,3-butadiene, and olefins). In some aspects, the partial pressure of H₂ used in the second step can range from 40% H₂ to 100% H₂, such as 50% H₂ to 100% H₂, or 60% H₂ to 100% H₂, or 70% H₂ to 100% H₂, or 80% H₂ to 100% H₂, or 90% H₂ to 100% H₂. In some examples, the partial pressure of H₂ was 67%, 80%, or 89%.

In particular aspects, the conversion of the acetaldehyde from the reaction product (including any unreacted acetaldehyde that might be recycled back to the second step) is carried out at a temperature ranging from 250° C. to 350° C., such as 275° C. to 350° C., or 300° C. to 350° C., or 300° C. to 325° C. and at a pressure ranging from 1 psig to 100 psig, such as 1 psig to 75 psig, or 1 psig to 50 psig. In some aspects, the temperature ranges from 275° C. to 325° C. and the pressure ranged from 35 psig to 50 psig. In some examples, the temperature was 300° C. and the pressure was 50 psig. In some examples, a weight hour space velocity (WHSV) ranging from 0.45 hr⁻¹ to 1.85 hr⁻¹, such as 0.47 hr⁻¹ to 1.85 hr⁻¹, 0.50 hr⁻¹ to 1.85 hr⁻¹, or 0.60 hr⁻¹ to 1.85 hr⁻¹, or 0.70 hr⁻¹ to 1.85 hr⁻¹, or 0.80 hr⁻¹ to 1.85 hr⁻¹, or 0.90 hr⁻¹ to 1.85 hr⁻¹, or 0.90 hr⁻¹ to 1.82 hr⁻¹. In some aspects, the WHSV ranges from 0.45 hr⁻¹ to 0.1.82 hr⁻¹, such as from 0.45 hr⁻¹ to 0.90 hr⁻¹. In some examples, the WHSV was 0.45, 0.90 hr⁻¹, or 1.85 hr⁻¹.

In particular aspects, the acetaldehyde from the reaction product is exposed to the second catalyst system in a second reactor operated at a temperature ranging from 250° C. to 350° C. and a pressure ranging from 1 psig to 100 psig. In particular aspects, the acetaldehyde from the reaction product is exposed to the second catalyst system in a second reactor operated at a temperature ranging from 250° C. to 350° C. In particular aspects, the acetaldehyde from the reaction product is exposed to the second catalyst system in a second reactor operated at a pressure ranging from 1 psig to 100 psig.

In some aspects of the disclosure, the second step of the method can result in conversion of a mixture comprising ethanol and acetaldehyde to 1,3-butadiene ranging from 30% to 90% or higher, such as 35% to 90%, or 40% to 90%, or 45% to 90%, or 50% to 90%, or 60% to 90% based on the amount of ethanol and acetaldehyde used as the feedstock for the second step of the method. In some aspects of the disclosure, the second step of the method can result in conversion of acetaldehyde to 1,3-butadiene ranging from 30% to 90% or higher, such as 35% to 90%, or 40% to 90%, or 45% to 90%, or 50% to 90%, or 60% to 90% based on the amount of acetaldehyde used as the feedstock for the second step of the method. In some aspects of the disclosure, the second step of the method can result in conversion of ethanol to 1,3-butadiene ranging from 30% to 90% or higher, such as 35% to 90%, or 40% to 90%, or 45% to 90%, or 50% to 90%, or 60% to 90% based on the amount of ethanol used as the feedstock for the second step of the method. In some aspects, the second step of the method can result in selectivities of 1,3-butadiene ranging from 50% to 100%, such as 60% to 100%, or 70% to 100%, or 80% to 100%.

### Overview of Several Aspects

Disclosed herein is a method for producing 1,3-butadiene from ethanol, the method comprising: exposing an ethanol-containing feedstock to a first catalyst system to produce a reaction product comprising acetaldehyde and H₂; and exposing the acetaldehyde of the reaction product to a second catalyst system to produce a 1,3-butadiene-containing composition, wherein exposing the acetaldehyde to the second catalyst system is done in the presence of at least a portion of the H₂ from the reaction product.

In any or all aspects of the disclosure, the acetaldehyde from the reaction product is exposed to the second catalyst system in a second reactor operated at a temperature ranging from 250° C. to 350° C. and a pressure ranging from 1 psig to 100 psig.

In any or all of the above aspects, the ethanol-containing feedstock is exposed to the first catalyst system in a first reactor operated at a temperature ranging from 300° C. to 450° C. and a pressure ranging from 1 psig to 200 psig.

In any or all of the above aspects, the acetaldehyde from the reaction product is exposed to the second catalyst system in a second reactor operated at a temperature ranging from 275° C. to 325° C. and a pressure ranging from 1 psig to 100 psig.

In any or all of the above aspects, the method further comprises isolating the H₂ from the reaction product.

In any or all of the above aspects, a portion of the H₂ from the reaction product is recycled and used in combination with the first catalyst system and/or the second catalyst system.

In any or all of the above aspects, the method further comprises isolating the H₂ from the 1,3-butadiene-containing composition.

In any or all of the above aspects, a portion of the H₂ from the 1,3-butadiene-containing composition is used in combination with the first catalyst system and/or the second catalyst system.

In any or all of the above aspects, the 1,3-butadiene-containing composition comprises unreacted acetaldehyde.

In any or all of the above aspects, the unreacted acetaldehyde is recycled and combined with the acetaldehyde of the reaction product.

In any or all of the above aspects, combined unreacted acetaldehyde and acetaldehyde of the reaction product are combined with the second catalyst system.

In any or all of the above aspects, the method further comprises combining ethanol with the unreacted acetaldehyde and/or the acetaldehyde of the reaction product.

In any or all of the above aspects, the ethanol is added in an amount that provides a ratio of ethanol:total acetaldehyde ranging from 1:1 to 3:1.

In any or all of the above aspects, combined unreacted acetaldehyde, acetaldehyde of the reaction product, and ethanol are combined with the second catalyst system.

In any or all of the above aspects, the first catalyst system comprises a metal and a support.

In any or all of the above aspects, the metal is copper and the support is an acidic support.

In any or all of the above aspects, the acidic support is SBA-16 or SiO₂ and wherein the first catalyst system further comprises a promoter selected from a Group I metal, a Row 4 metal, or a combination thereof.

In any or all of the above aspects, the second catalyst system comprises a zirconium oxide, a second catalyst support, and a metal dopant.

In any or all of the above aspects, the second catalyst support is acidic.

In any or all of the above aspects, the second catalyst support is a mesoporous SiO₂ material and the metal dopant is silver.

In any or all of the above aspects, the second catalyst comprises 0.5 wt % to 8 wt % Ag and 2 wt % to 12 wt % ZrO₂.

In any or all of the above aspects, exposing the ethanol-containing feedstock to the first catalyst system is carried out at a temperature ranging from 375° C. to 425° C. and a pressure ranging from 50 psig to 200 psig; and exposing the acetaldehyde of the reaction product to the second catalyst system is carried out at a temperature ranging from 300° C. to 325° C. and a pressure ranging from 35 psig to 50 psig.

In any or all of the above aspects, the method further comprises performing a purification step.

In any or all aspects of the disclosure, the method comprises: exposing an ethanol-containing feedstock to a first catalyst system in a first reactor to produce a reaction product comprising acetaldehyde and H₂; passing the acetaldehyde of the reaction product to a second reactor; passing at least a portion of H₂ of the reaction product to the second reactor; and exposing the acetaldehyde of the reaction product to a second catalyst system in the second reactor to produce a 1,3-butadiene-containing composition, wherein exposing the acetaldehyde to the second catalyst is done in the presence of the H₂ from the reaction product.

In any or all of the above aspects, (i) the first catalyst comprises chromium, copper, and a silica support, wherein the copper and chromium are present in amounts that provide a ratio of Cu:Cr ranging from 10:1 to 10:3; (ii) the second catalyst comprises 0.5 wt % to 8 wt % Ag, 2 wt % to 12 wt % ZrO₂, and mesoporous SiO₂ material; (iii) the method further comprises recycling a portion of the H₂ from the reaction product to the first reactor; (iv) the method further comprises recycling any unreacted acetaldehyde from the second reactor by passing the unreacted acetaldehyde back to the second reactor; (v) the method further comprises adding ethanol to the second reactor; or (vi) any combination of two or more of (i) to (v).

In any or all of the above aspects, the first reactor is operated at a temperature ranging from 350° C. to 425° C. and a pressure of 1 psig to 200 psig; and the second reactor is operated at a temperature of 275° C. to 325° C. and a pressure of 1 psig to 75 psig.

In any or all of the above aspects, ethanol is added to the second reactor.

In any or all of the above aspects, the ethanol is added to the second reactor simultaneously with the acetaldehyde from the reaction product and the ethanol and acetaldehyde are introduced to the second reactor at a ratio ranging from 1:1 to 3:1 (ethanol:acetaldehyde).

### EXAMPLES

General procedure for evaluating first step (ethanol-containing feedstock to acetaldehyde) conversion parameters: Reactivity evaluations for converting ethanol to acetaldehyde were conducted in a 6.35-mm outer diameter (inner diameter=4.57 mm) fixed-bed, packed-bed reactor loaded with 2.0 g of a first catalyst system. A K-type thermocouple was placed in the reactor to measure the catalyst bed temperature. To minimize temperature gradients, an electrical resistance heating block was installed on the reactor. Prior to testing, the first catalyst system was first activated in situ at 450° C. for 8 hours under 120 SCCM of N₂. Then, the temperature was cooled to 325° C. and the first catalyst system was reduced under 100 SCCM of 10% H₂/N₂ for 1 hour. Ethanol was fed into the system using an ISCO syringe pump and was converted to the gas phase using a vaporizer comprising a 6.6 nm inner diameter steel tubing filled with quartz beads. A knockout pot placed directly downstream of the reaction zone was used to collect liquid product. Gaseous effluent was analyzed online using an Inficon micro-GC (Model 3000A) equipped with MS-5A, Plot U, alumina, OV-1 columns, and a thermal conductivity detector. Liquid samples were collected from the knockout pot were analyzed separately ex situ using liquid chromatography.

General procedure for evaluating second step (acetaldehyde to 1,3-butadiene) conversion parameters: Reactivity evaluations for converting ethanol and acetaldehyde to 1,3-butadiene were conducted in a 6.35-mm outer diameter (inner diameter=4.57 mm) fixed-bed, packed-bed reactor loaded with 2.0 g of a second catalyst system. A K-type thermocouple was placed in the reactor to measure the catalyst bed temperature. To minimize temperature gradients, an electrical resistance heating block was installed on the reactor. Prior to testing, the second catalyst system was first activated in situ at 450° C. for 8 hours under 120 SCCM of N₂. Then, the temperature was cooled to 325° C. and the catalysts was reduced under 100 sccm of 10% H₂/N₂ for 1 hour. Ethanol and acetaldehyde were fed into the system at a 2:1 ratio (unless indicated otherwise in examples herein) using an ISCO syringe pump were converted to the gas phase using a vaporizer consisting of 6.6 nm inner diameter steel tubing filled with quartz beads. A knockout pot placed directly downstream of the reaction zone was used to collect liquid product. Gaseous effluent was analyzed online using an Inficon micro-GC (Model 3000A) equipped with MS-5A, Plot U, alumina, OV-1 columns, and a thermal conductivity detector. Liquid samples were collected from the knockout pot were analyzed separately ex situ using liquid chromatography.

### Example 1

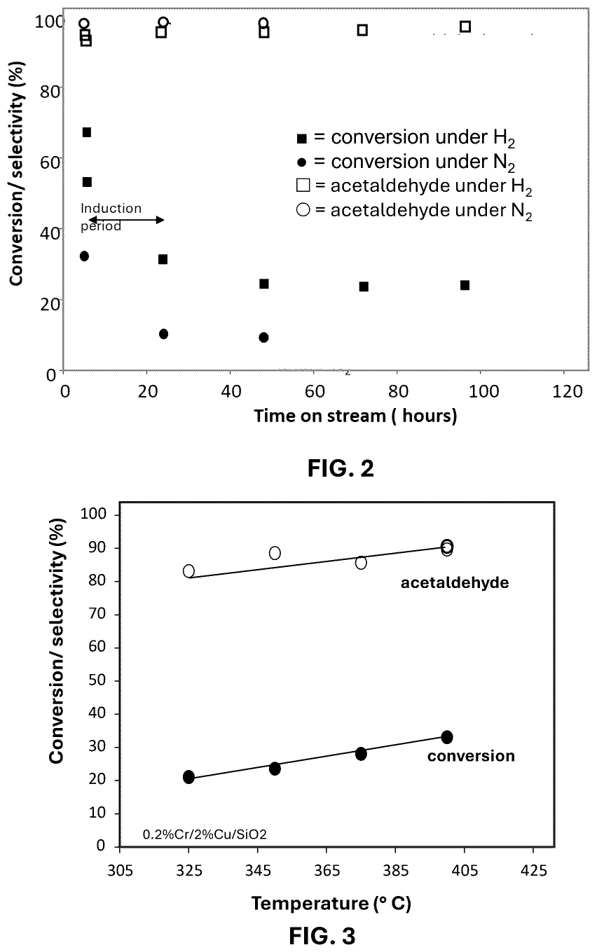

In this example, the effects of using a H₂-rich atmosphere for the first step (conversion of ethanol to acetaldehyde) of the disclosed method on conversion and selectivity for acetaldehyde was evaluated. The first catalyst system that was used was 2% Cu/SBA-16. Conversion and selectivity values were measured after converting an ethanol-containing feedstock to acetaldehyde using a reaction temperature of 400° C. at 7 atmospheres, with a 11% P_{EtOH} feed, with the balance being either H₂ or N₂ at 6.6 hr⁻¹ WHSV, with TOS (time on stream) of up to 100 hours. Results are shown in FIG. 2 and summarized in Table 1 below. As can be seen in FIG. 2, ethanol dehydrogenation to acetaldehyde was favored over undesired ethanol dehydration to ethylene and diethyl ether. High conversion values were observed using the H₂-rich environment, showing that catalyst stability and activity can be increased in an H₂-rich environment as compared with an inert environment (e.g., N₂).

TABLE 1
Conversion and acetaldehyde selectivity for step 1 when
operating under H₂ or N₂ environment over 2% Cu/SBA-
16. T = 400° C., P = 7 atmospheres, WHSV = 6.6 hr⁻¹.

| | | | Acetaldehyde |
|---|---|---|---|
| | Time on stream | Conversion | selectivity |
| | (hours) | (%) | (%) |
| Under H₂ | 5 | 53.1 | 93.5 |
| | 24 | 31.1 | 97.9 |
| | 48 | 24.6 | 98.5 |
| | 72 | 23.8 | 96.5 |
| | 96 | 24.1 | 96.8 |
| Under N₂ | 5 | 32.2 | 97.9 |
| | 24 | 10.2 | 98.3 |
| | 48 | 9.2 | 98.0 |

### Example 2

In this example, the effects of reaction temperature were evaluated for the first step of converting ethanol to acetaldehyde. The first catalyst system that was used was 0.2% Cr/2% Cu/SiO₂ or 2% Cu/SBA-16. Conversion and selectivity values were measured after converting an ethanol-containing feedstock to acetaldehyde using reaction temperatures ranging from 325° C. to 400° C. (namely 325° C., 350° C., 375° C., and 400° C.) at 7 atmospheres, with a 11% P_{EtOH} feed, with the balance being H₂ at 1.66 hr⁻¹ WHSV with a TOS of 48 hours. Results for this example are shown in FIG. 3 and summarized in Table 2 below. It was observed that good conversion and selectivity could be achieved in this example by increasing the temperature to 400° C., both for the 0.2% Cr/2% Cu/SiO₂ catalyst system and the 2% Cu/SBA-16 (results not shown in FIG. 3).

TABLE 2
Conversion and acetaldehyde selectivity for step 1 over 0.2% Cr/2%
Cu/SiO₂. T = 325-400° C., P = 7 atmospheres, WHSV = 1.66 hr⁻¹.

| Temperature | Conversion | Acetaldehyde Selectivity |
|---|---|---|
| (° C.) | (%) | (%) |
| 325 | 21.1 | 83.2 |
| 350 | 23.6 | 88.6 |
| 375 | 28.1 | 85.7 |
| 400 | 33.1 | 89.7 |

### Example 3

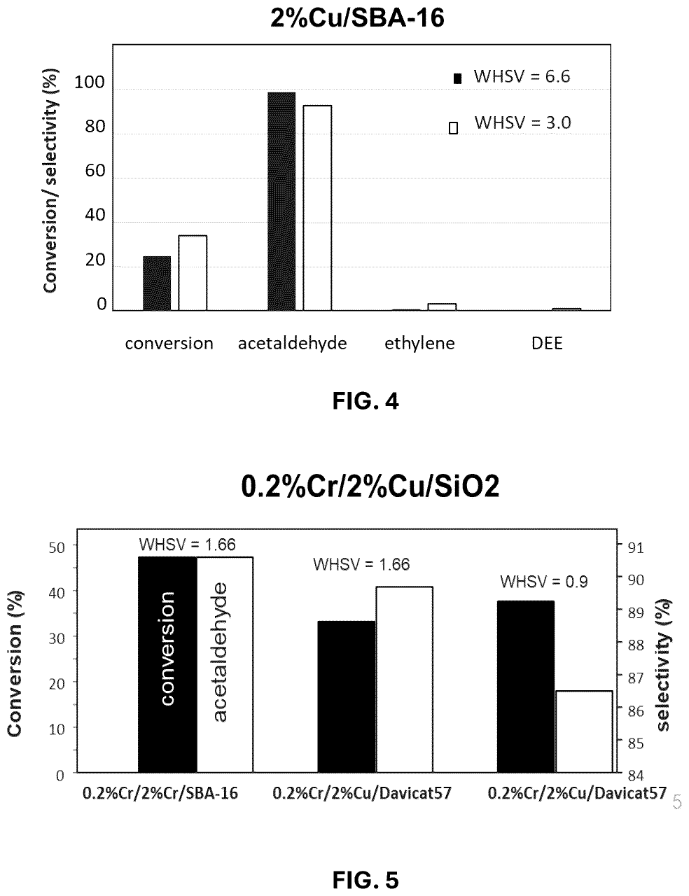

In this example, the effects of WHSV were evaluated for the first step of converting ethanol to acetaldehyde. The first catalyst system that was used was 0.2% Cr/2% Cu/SiO₂ or 2% Cu/SBA-16. Conversion and selectivity values were measured after converting an ethanol-containing feedstock to acetaldehyde using a reaction temperature of 400° C. at 7 atmospheres, with a 11% P_{EtOH} feed, with the balance being H₂, and at different WHSV values (namely 0.9 hr⁻¹, 1.66 hr⁻¹, 3.0 hr⁻¹, and 6.6 hr⁻¹) with a TOS ranging from 48-96 hours. Results for this example are shown in FIGS. 4 and 5 and summarized in Table 3 below. In this example, it was observed that higher contact times and/or lower space velocities provided higher conversion rates for some examples; however, in some examples, the selectivity to desired acetaldehyde decreased, potentially due to secondary reactions (e.g., formation of ethyl acetate, butyraldehyde).

TABLE 3
Conversion and selectivities toward acetaldehyde, ethylene, and diethyl
ether for step 1 over 2% Cu/SBA-16, 0.2% Cr/2% Cu/SBA-16, and 0.2% Cr/2%
Cu/SiO₂. T = 400° C., P = 7 atmospheres, WHSV = 0.9-6.6 hr⁻¹.

| | | | Selectivity (%) | | |
|---|---|---|---|---|---|
| | | | | | Diethyl |
| | WHSV | Conversion | | | ether |
| Catalyst | (hr⁻¹) | (%) | Acetaldehyde | ethylene | (DEE) |
| 2% Cu/ SBA-16 | 6.6 | 24.1 | 96.8 | 0.6 | 0.2 |
| | 3.0 | 28.3 | 92.3 | 3.1 | 0.7 |
| 0.2% Cr/2% Cu/SBA-16 | 1.66 | 47.2 | 90.6 | 1.6 | 0.3 |
| 0.2% Cr/2% Cu/SiO₂ | 1.66 | 33.1 | 89.7 | 0.5 | 0.0 |
| 0.2% Cr/2% Cu/SiO₂ | 0.9 | 37.5 | 86.5 | 0.7 | 0.0 |

### Example 4

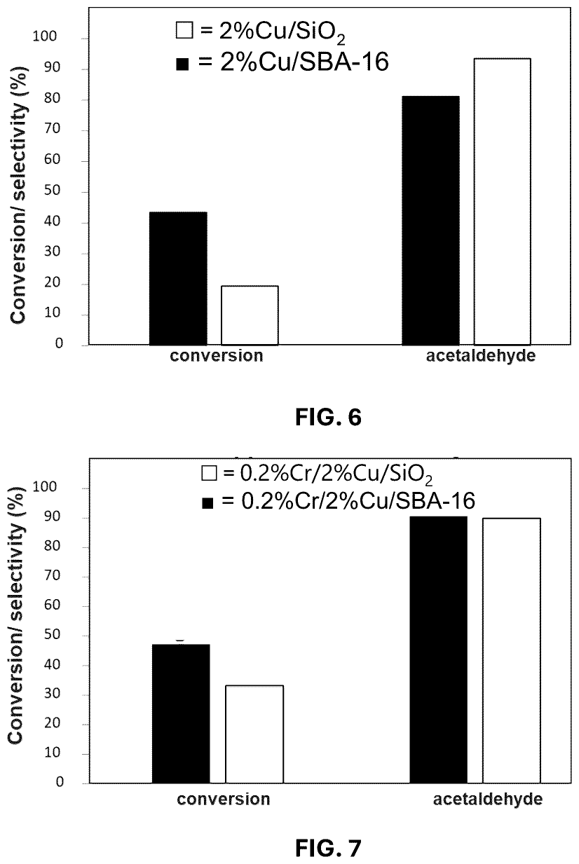

In this example, the effect of acidic support surface area was evaluated for the first step of converting ethanol to acetaldehyde. The first catalyst system that was used was either 0.2% Cr/2% Cu/SiO₂ or 2% Cu/SBA-16. Conversion and selectivity values were measured after converting an ethanol-containing feedstock to acetaldehyde using a reaction temperature of 400° C. at 7 atmospheres, with a 11% P_{EtOH} feed, with the balance being H₂, and at a WHSV value of 1.66 hr⁻¹ with a TOS of 48 hours. Results for this example are shown in FIGS. 6 and 7, and summarized in Table 4 below. In this example, it was observed that the SBA-16 acidic support could provide larger surface area and facilitate higher conversion rates.

TABLE 4
Conversion and acetaldehyde selectivity for step 1 over 2%
Cu/SBA-16, 2% Cu/SiO₂, 0.2% Cr/2% Cu/SBA-16, and 0.2% Cr/2%
Cu/SiO₂. T = 400° C., P = 7 atmospheres, WHSV = 1.66 hr⁻¹.

| Catalyst | Conversion (%) | Acetaldehyde selectivity (%) |
|---|---|---|
| 2% Cu/SBA-16 | 43.3 | 81.0 |
| 2% Cu/SiO₂ | 19.3 | 93.4 |
| 0.2% Cr/2% Cu/SBA-16 | 47.2 | 90.6 |
| 0.2% Cr/2% Cu/SiO₂ | 33.1 | 89.7 |

### Example 5

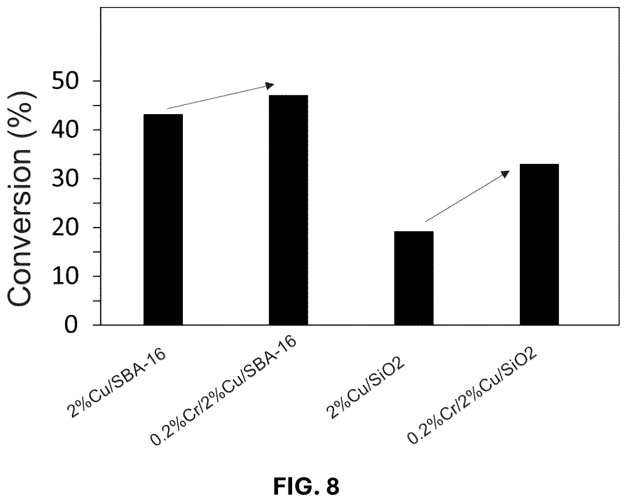

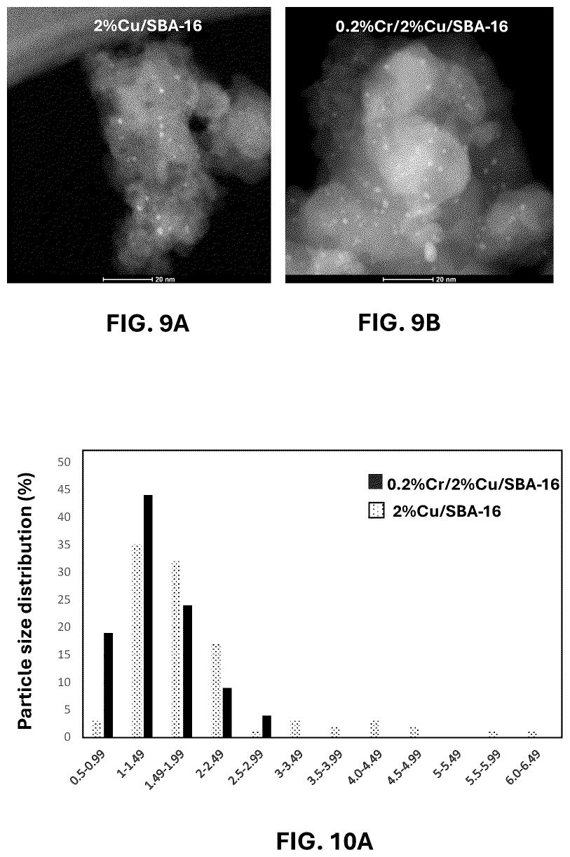

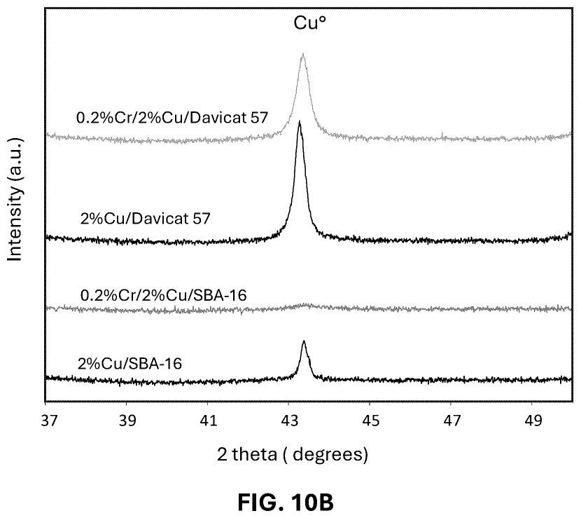

In this example, the effect of using a metal promoter was evaluated for the first step of converting ethanol to acetaldehyde. The first catalyst system that was used included: 0.2% Cr/2% Cu/SiO₂, 2% Cu/SiO₂, 0.2% Cr/2% Cu/SBA-16, and 2% Cu/SBA-16. Conversion and selectivity values were measured after converting an ethanol-containing feedstock to acetaldehyde using a reaction temperature of 400° C. at 7 atmospheres, with a 11% P_{EtOH} feed, with the balance being H₂, and at a WHSV value of 1.66 hr⁻¹ with a TOS of 48 hours. Results for this example are shown in FIG. 8 and summarized in Table 5 below. In this example, it was observed that conversion rates and acetaldehyde selectivity could be increased by adding a metal promoter, particularly chromium, to the first catalyst system. The ratio of Cu to Cr was 10:1. Also, the promoter can be used to influence dispersion of the metal into the support. For example, FIGS. 9A and 9B show that 2% Cu/SBA-16 and 0.2% Cr/2% Cu/SBA-16 have small particles with an average particle size of 2% Cu/SBA-16=1.9 nm and 0.2% Cr/2% Cu/SBA-16=1.4 nm. And, FIGS. 10A and 10B show (i) a particle size distribution graph, which establishes that the majority of the particles are between 1-1.5 nm for both catalysts (FIG. 10A) and (ii) an XRD spectrum showing that Cr improves dispersion.

TABLE 5
Conversion for step 1 over 2% Cu/SBA-16, 2%
Cu/SiO₂, 0.2% Cr/2% Cu/SBA-16, and 0.2% Cr/2%
Cu/SiO₂. T = 400° C., P = 7 atmospheres, WHSV = 1.66 hr⁻¹.

| Catalyst | Conversion (%) |
|---|---|
| 2% Cu/SBA-16 | 43.3 |
| 0.2% Cr/2% Cu/SBA-16 | 47.2 |
| 2% Cu/SiO₂ | 19.3 |
| 0.2% Cr/2% Cu/SiO₂ | 33.1 |

### Example 6

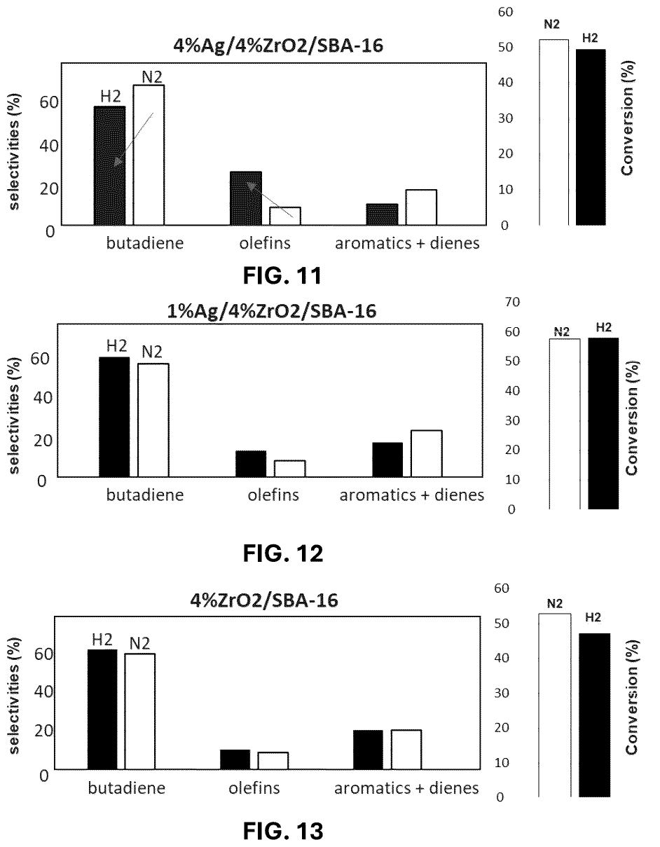

In this example, the effects of using a H₂-rich atmosphere for the second step (conversion of acetaldehyde to 1,3-butadiene) of the disclosed method on conversion and selectivity for 1,3-butadiene was evaluated. Three different second catalyst systems were evaluated: 4% Ag/4% ZrO₂/SBA-16, 1% Ag/4% ZrO₂/SBA-16, and 4% ZrO₂/SBA-16. Conversion and selectivity values were measured after converting an acetaldehyde feedstock, with a co-feed comprising ethanol and H₂, to 1,3-butadiene using a reaction temperature of 325° C. at 7 atmospheres, with a feed comprising EtOH and acetaldehyde at a ratio of 2:1, using a WHSV value of 1.8 hr⁻¹ and either an H₂-rich atmosphere, or under inert gas (N₂). Results for this example are shown in FIGS. 11-13 and summarized in Table 6 below. In this example, it was observed that the 1% Ag/4% ZrO₂/SBA-16 catalyst system exhibited higher butadiene selectivity than the system comprising 4% Ag under H₂, potentially owing to limited hydrogenation to butenes with this system. In this example, the 1% Ag/4% ZrO₂/SBA-16 presents slightly higher conversion and lower aromatics formation as compared to 4% ZrO₂/SBA-16, though the 4% ZrO₂/SBA-16 catalyst still provided good conversion values.

TABLE 6
Effect of the environment (i.e., H₂ or N₂) on the conversion and
selectivity toward butadiene, olefins, aromatics + dienes for step 2 over
4% Ag/4% ZrO₂/SBA-16, 1% Ag/4% ZrO₂/SBA-16, and 4% ZrO₂/SBA-16. T =
325° C., P = 7 atmospheres, WHSV = 1.8 hr⁻¹.

| | | | Selectivity (%) | | |
|---|---|---|---|---|---|
| | H₂ or N₂ | Conversion | | | Aromatics + |
| Catalyst | environment | (%) | butadiene | olefins | dienes |
| 4% Ag/4% ZrO₂/SBA-16 | H₂ | 49.4 | 54.8 | 24.6 | 9.6 |
| | N₂ | 52.2 | 64.9 | 8.3 | 16.4 |
| 1% Ag/4% ZrO₂/SBA-16 | H₂ | 57.9 | 61.5 | 13.6 | 17.8 |
| | | | 58.1 | 8.3 | 23.9 |
| 4% ZrO₂/SBA-16 | H₂ | 47.1 | 61.8 | 10.2 | 20.2 |
| | N₂ | 52.8 | 59.4 | 8.7 | 20.2 |

### Example 7

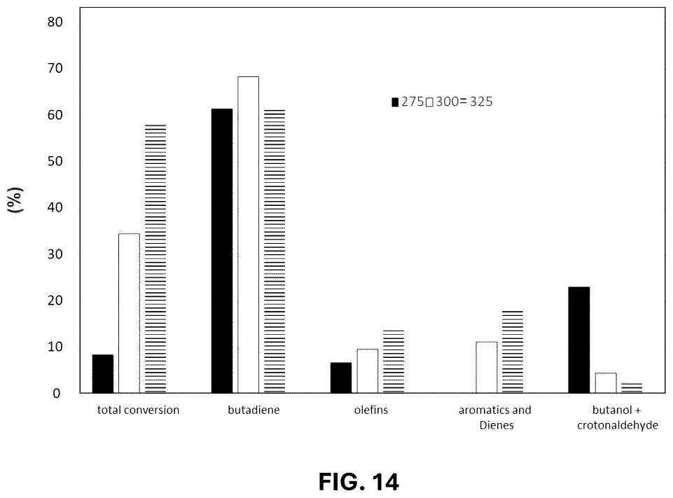

In this example, the effects of reaction temperature were evaluated for the second step of converting acetaldehyde to 1,3-butadiene. The second catalyst system that was used was 1% Ag/4% ZrO₂/SBA-16. Conversion and selectivity values were measured after converting an acetaldehyde-containing feedstock to 1,3-butadiene using reaction temperatures ranging from 275° C. to 325° C. (namely 275° C., 300° C., and 325° C.) at 7 atmospheres, with a feed comprising EtOH and acetaldehyde at a ratio of 2:1, using a WHSV value of 1.8 hr⁻¹ and an H₂-rich atmosphere. Results for this example are shown in FIG. 14 and summarized in Table 7 below. In this example, it was observed that the lower temperature inhibited butadiene hydrogenation to butene and dimerization cyclization to aromatics, to some extent. In this example, a 68% butadiene selectivity obtained at 300° C. with conversion of 34%.

TABLE 7
Conversion and selectivity for step 2 over 1% Ag/4% ZrO₂/SBA-16 at varied
temperatures between 275-325° C. P = 7 atmospheres, WHSV = 1.8 hr⁻¹.

| | | Selectivity (%) | | | |
|---|---|---|---|---|---|
| Temperature | Conversion | | | aromatics + | butanol + |
| (° C.) | (%) | butadiene | olefins | dienes | crotonaldehyde |
| 275 | 8.3 | 61.3 | 6.6 | 0 | 22.9 |
| 300 | 34.4 | 68.3 | 9.5 | 11.1 | 4.4 |
| 325 | 57.9 | 61.5 | 13.6 | 17.8 | 2.1 |

### Example 8

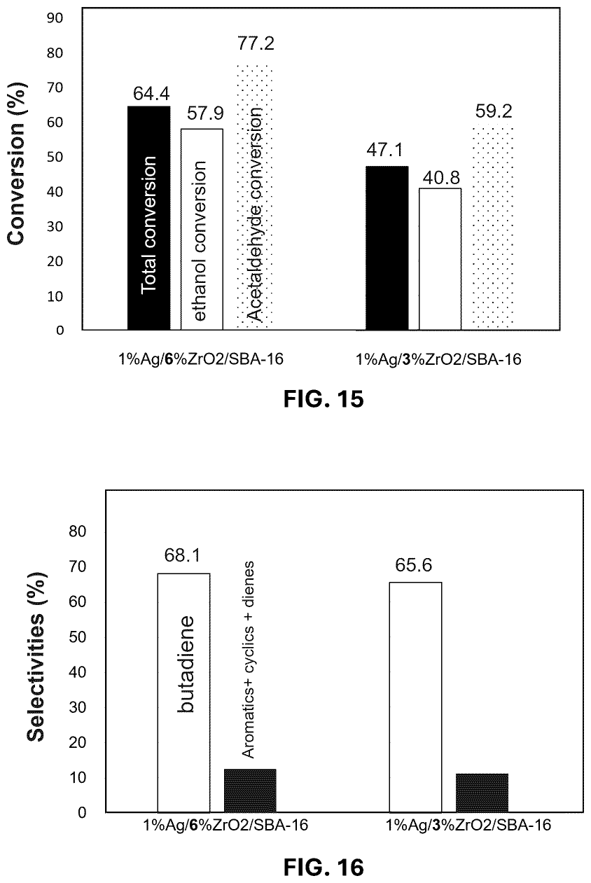

In this example, the effects of ZrO₂ loading were evaluated for the second step of converting acetaldehyde to 1,3-butadiene. The second catalyst system was designed to comprise 1% Cr and varying amounts of ZrO₂ loading on the acidic substrate (SBA-16). Specific catalyst systems included 1% Ag/6% ZrO₂/SBA-16 and 1% Ag/3% ZrO₂/SBA-16. Conversion and selectivity values were measured after converting an acetaldehyde-containing feedstock to 1,3-butadiene using a reaction temperature of 300° C. at 3.5 atmospheres, with a feed comprising EtOH and acetaldehyde at a ratio of 2:1, using a WHSV value of 0.9 hr⁻¹ and an H₂-rich atmosphere (P_{H2}/P_{feed}=89/11). Results for this example are shown in FIGS. 15 and 16, and summarized in Table 8 below. In this example, it was observed that the conversion was higher with the 6% ZrO₂ catalyst (giving a conversion value of 64.4%) as compared with the 3% ZrO₂ catalyst (giving a conversion value of 47.1%). The butadiene selectivity was also higher in this example for the catalyst with 6% ZrO₂ (giving a value of 68.1%).

TABLE 8
Effect of ZrO₂ loading on the performance of 1% Ag/x % ZrO₂/SBA-16
for step 2. T = 300° C., P = 3.5 atmospheres, WHSV = 0.9 hr⁻¹.

| | | | | Selectivity (%) | |
|---|---|---|---|---|---|
| ZrO₂ | Total | ethanol | Acetaldehyde | | aromatics + |
| loading | conversion | conversion | conversion | | cyclics + |
| (%) | (%) | (%) | (%) | butadiene | dienes |
| 3 | 47.1 | 40.8 | 59.2 | 65.6 | 11.2 |
| 6 | 64.4 | 57.9 | 77.2 | 68.1 | 12.5 |

### Example 9

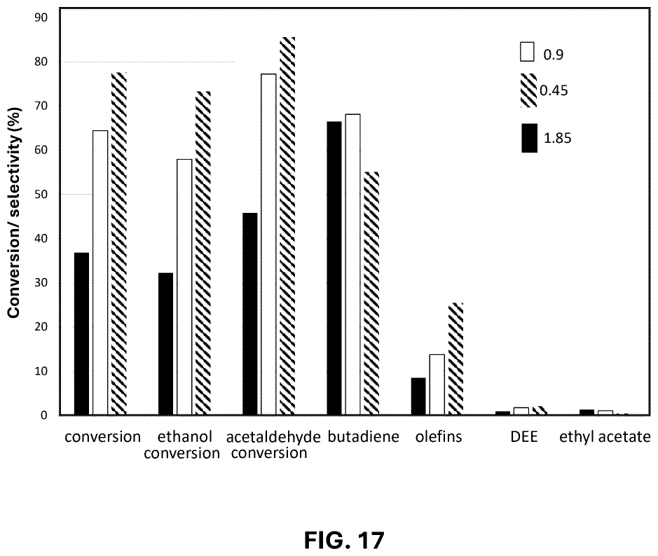

In this example, the effects of WHSV at 3.5 atmospheres (50 psig) was evaluated for the second step of converting acetaldehyde to 1,3-butadiene. The second catalyst system that was used was 1% Ag/6% ZrO₂/SBA-16. Conversion and selectivity values were measured after converting an acetaldehyde-containing feedstock to 1,3-butadiene using a reaction temperature of 300° C. at 3.5 atmospheres, with a feed comprising EtOH and acetaldehyde at a ratio of 2:1, using varying WHSV values (namely 0.47-1.85 hr⁻¹) and an H₂-rich atmosphere. Results for this example are shown in FIG. 17 and summarized in Table 9 below. In this example, it was observed that decreasing the WHSV from 1.82 to 0.45 leads to an increase in conversion, with the ethanol conversion increasing from 32.2% to 73.3% and the acetaldehyde conversion increasing from 45.8 to 85.5%. Butadiene selectivity decreased at the lowest WHSV (i.e., 0.45 hr⁻¹) and was observed to be 55%. A butadiene selectivity equal to 68.1% at high acetaldehyde conversion (77.2%) and ethanol conversion of 57.9% was achieved at 50 psig and WHSV=0.9 hr⁻¹.

TABLE 9
Effect of the WHSV on the catalytic performance for step 2 over 1% Ag/6% ZrO₂/SBA-16.
T = 300° C., = 3.5 WHSV = 1.85 hr⁻¹.

| | | | | Selectivities (%) | | | |
|---|---|---|---|---|---|---|---|
| | Total | Ethanol | Acetaldehyde | | | Diethyl | |
| WHSV | conversion | conversion | conversion | | | ether | Ethyl |
| (hr⁻¹) | (%) | (%) | (%) | butadiene | olefins | (DEE) | acetate |
| 0.45 | 77.5 | 73.3 | 85.5 | 66.5 | 8.5 | 0.9 | 1.3 |
| 0.9 | 64.4 | 57.9 | 77.2 | 68.1 | 13.7 | 1.7 | 1.0 |
| 1.85 | 36.8 | 32.2 | 45.8 | 55.0 | 25.4 | 2.0 | 0.4 |

### Example 10

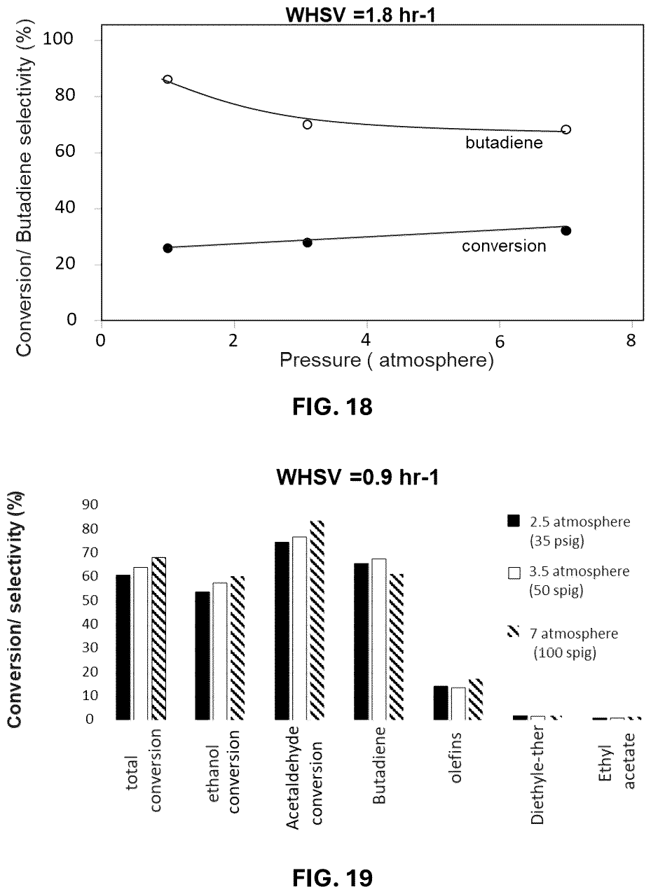

In this example, the effect of modifying pressure at a WHSV of 0.9 hr⁻¹ or 1.8 hr⁻¹ was evaluated for the second step of converting acetaldehyde to 1,3-butadiene. The second catalyst system that was used was 1% Ag/6% ZrO₂/SBA-16. Conversion and selectivity values were measured after converting an acetaldehyde-containing feedstock to 1,3-butadiene using a reaction temperature of 300° C. at varying pressures ranging from 1 atmospheres to 7 atmospheres. In some examples, the pressure ranges from 1, 3, and 7 atmospheres, with a feed comprising EtOH and acetaldehyde at a ratio of 2:1, using a WHSV of 1.8 hr⁻¹ and an H₂-rich atmosphere (100% H₂, 11% ethanol+acetaldehyde). In some examples, the pressure ranges from 2.5, 3.5, and 7 atmospheres, with a feed comprising EtOH and acetaldehyde at a ratio of 2:1, using a WHSV of 0.9 hr⁻¹ and an H₂-rich atmosphere (100% H₂, 11% ethanol+acetaldehyde. Results are shown in FIGS. 18 and 19, Table 10 below. In this example, it was observed that decreasing the pressure resulted in decreasing the conversion. 1,3-butadiene selectivity of 68% was observed, along with an acetaldehyde conversion of 77.2%.

TABLE 10
Effect of the pressure on the catalytic performance for step 2 over 1% Ag/6%
ZrO₂/SBA-16. T = 300° C., WHSV = 0.9 and 1.8 hr⁻¹.

| | | Total | Ethanol | Acetaldehyde | Selectivity (%) | | | |
|---|---|---|---|---|---|---|---|---|
| WHSV | Pressure | conversion | conversion | conversion | | | Diethyl | Ethyl |
| (hr⁻¹) | (atmospheres) | (%) | (%) | (%) | butadiene | olefins | ether | acetate |
| 1.8 | 1 | 25.9 | 21.9 | 31.3 | 86.1 | 6.6 | 1.9 | 0.9 |
| | 3 | 27.9 | 22.2 | 36.7 | 69.9 | 8.1 | 1.6 | 1.0 |
| | 7 | 32.2 | 27.5 | 41.9 | 68.3 | 10.2 | 0.0 | 3.4 |
| 0.9 | 2.5 | 61.1 | 53.9 | 74.9 | 66.0 | 14.5 | 2.0 | 1.0 |
| | 3.5 | 64.4 | 57.9 | 77.2 | 68.1 | 13.9 | 1.7 | 1.0 |
| | 7.0 | 68.4 | 60.5 | 83.8 | 61.4 | 17.4 | 1.8 | 1.4 |

### Example 11

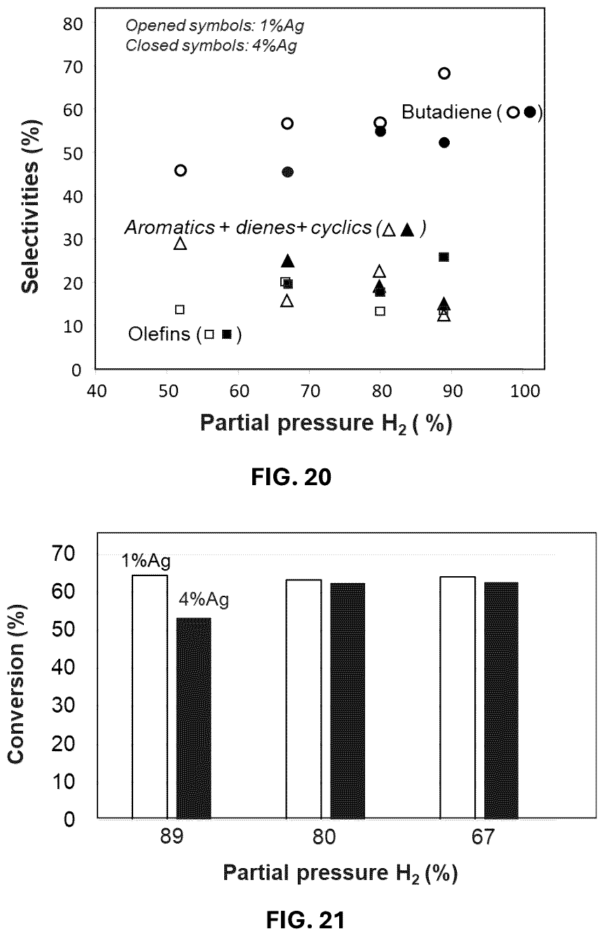

In this example, the effect of modifying H₂ partial pressure at constant WHSV was evaluated for the second step of converting acetaldehyde to 1,3-butadiene. The second catalyst systems that were used included 1% Ag/6% ZrO₂/SBA-16 and 4% Ag/6% ZrO₂/SBA-16. Conversion and selectivity values were measured after converting an acetaldehyde-containing feedstock to 1,3-butadiene using a reaction temperature of 300° C. at varying H₂ partial pressures ranging from 67% to 89% (namely, 67%, 80%, and 89% H₂), with a feed comprising EtOH and acetaldehyde at a ratio of 2:1, using a WHSV of 0.9 hr⁻¹ and a pressure of 3.5 atmospheres. Results for this example are shown in FIGS. 20 and 21, and summarized in Table 11 below. In this example, it was observed that increasing the H₂ partial pressure can facilitate increasing conversion to 1,3-butadiene with increased selectivity. For this example, the lower Ag loading of 1% performed better than the 4% Ag-containing catalyst system.

TABLE 11
Effect of H₂ partial pressure on the catalytic performance
for 1% Ag/6% ZrO₂/SBA-16 and 4% Ag/6% ZrO₂/SBA-16. T =
300° C., WHSV = 0.9 hr^{-1,} P = 3.5 atmospheres.

| | | | Selectivity (%) | | |
|---|---|---|---|---|---|
| | Partial | | | | Aromatics + |
| Ag loading | pressure H₂ | Conversion | | | dienes + |
| (%) | (%) | (%) | butadiene | olefins | cyclics |
| 1 | 52 | 66.5 | 45.7 | 14.1 | 28.6 |
| | 67 | 64.0 | 56.5 | 19.2 | 15.2 |
| | 80 | 63.2 | 56.8 | 12.9 | 22.4 |
| | 89 | 64.4 | 68.1 | 13.7 | 12.5 |
| 4 | 67 | 62.7 | 45.3 | 19.5 | 20.9 |
| | 80 | 62.5 | 54.8 | 17.4 | 14.5 |
| | 89 | 53.2 | 52.0 | 25.4 | 11.4 |

### Example 12

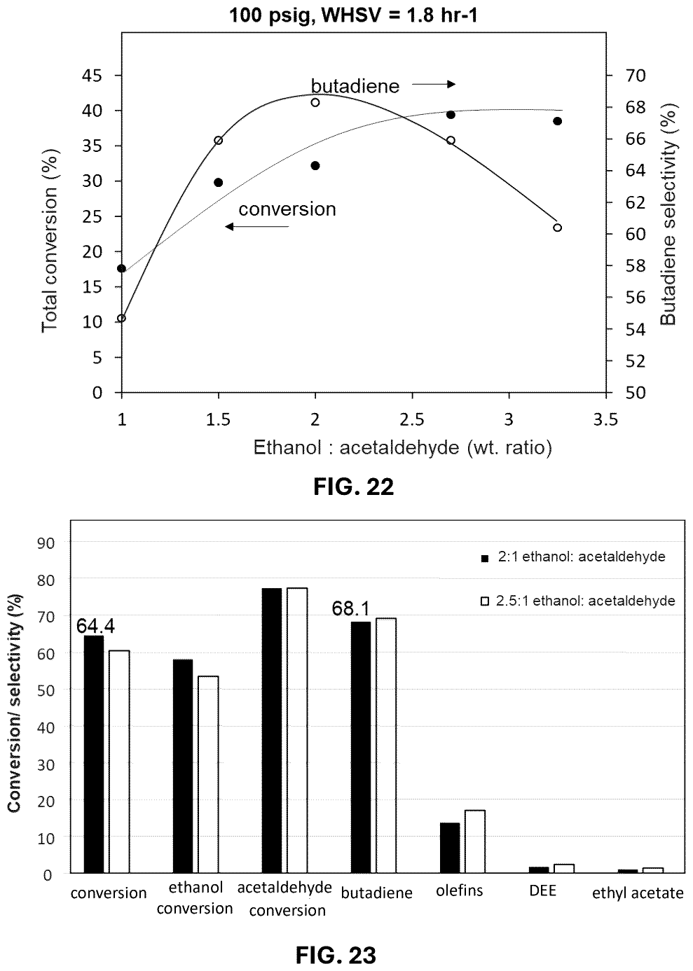

In this example, the effect of modifying the feedstock composition was evaluated for the second step of converting acetaldehyde to 1,3-butadiene. The second catalyst systems that was used was 1% Ag/6% ZrO₂/SBA-16. Conversion and selectivity values were measured after converting an acetaldehyde-containing feedstock to 1,3-butadiene using a reaction temperature of 300° C. and a feedstock composition comprising EtOH and acetaldehyde at ratios ranging from 1:1 to 3.3:1 EtOH:acetaldehyde (namely, 1:1, 1.5:1, 2.0:1, 2.7:1, and 3.3:1) at 7 atmospheres using a WHSV of 1.8 hr⁻¹, with 11% P_{EtOH}+P_{acetaldehyde}, balance H₂. Results for this example are shown in FIG. 22 and summarized in Table 12. In this example, it was observed that conversion increases with increases in the EtOH:acetaldehyde ratio and 1,3-butadiene selectivity can be affected by the ratio, with some examples giving more olefin selectivity upon higher amounts of ethanol in the feed. In this example, the highest 1,3-butadiene selectivity of 68.3% was obtained for an ethanol:acetaldehyde ratio of 2:1, with a conversion of 32.2% (FIG. 22).

TABLE 12
Effect of the ethanol:acetaldehyde ratio (in weight) on the
conversion and butadiene selectivity for 1% Ag/6% ZrO₂/SBA-16
at T = 300° C., P = 7 atmospheres, WHSV = 1.8 hr⁻¹.

| ethanol:acetaldehyde | conversion (%) | butadiene selectivity (%) |
|---|---|---|
| 1:1 | 17.6 | 54.7 |
| 1.5:1 | 29.8 | 65.9 |
| 2.0:1 | 32.2 | 68.3 |
| 2.7:1 | 39.4 | 65.9 |
| 3.3:1 | 38.5 | 60.4 |

### Example 13

In this example, the effect of modifying the feedstock composition was evaluated for the second step of converting acetaldehyde to 1,3-butadiene. The second catalyst system that was used was 1% Ag/6% ZrO₂/SBA-16. Conversion and selectivity values were measured after converting an acetaldehyde-containing feedstock to 1,3-butadiene using a reaction temperature of 300° C. and a feedstock composition comprising EtOH and acetaldehyde at ratios ranging from 1:1 to 2.7:1 EtOH:acetaldehyde (namely, 2:1 and 2.5:1) at 3.5 atmospheres using a WHSV of 0.9 hr⁻¹, with 11% P_{EtOH}+P_{acetaldehyde}, balance H₂. Results for this example are shown in FIG. 23 and summarized in Table 13. In this example, it was observed that conversion increases with increases in the EtOH:acetaldehyde ratio and 1,3-butadiene selectivity can be affected by the ratio, with some examples giving more olefin selectivity upon higher amounts of ethanol in the feed. In this example, the highest 1,3-butadiene selectivity of 68.1% was obtained for an ethanol:acetaldehyde ratio of 2:1, with conversion of 64.4%.

TABLE 13
Effect of the ethanol:acetaldehyde ratio (in weight) on the conversion and butadiene selectivity
for 1% Ag/6% ZrO₂/SBA-16 at T = 300° C., P = 3.5 atmospheres, WHSV = 0.9 hr⁻¹.

| | Total | Ethanol | Acetaldehyde | Selectivity (%) | | | |
|---|---|---|---|---|---|---|---|
| | conversion | conversion | conversion | | | Diethyl | Ethyl |
| Ethanol:acetaldehyde | (%) | (%) | (%) | butadiene | olefins | ether | acetate |
| 2:1 | 64.4 | 57.9 | 77.2 | 68.1 | 13.7 | 1.7 | 1.0 |
| 2.5:1 | 60.3 | 53.3 | 77.3 | 69.0 | 25.4 | 2.3 | 1.4 |

In view of the many possible embodiments to which the principles of the present disclosure may be applied, it should be recognized that the illustrated embodiments are only preferred examples of the disclosure and should not be taken as limiting the scope of the disclosure. Rather, the scope is defined by the following claims. We therefore claim as our invention all that comes within the scope and spirit of these claims.

We claim:

1. A method for producing 1,3-butadiene from ethanol, the method comprising:
exposing an ethanol-containing feedstock to a first catalyst system to produce a reaction product comprising acetaldehyde and H₂; and
exposing the acetaldehyde of the reaction product to a second catalyst system to produce a 1,3-butadiene-containing composition, wherein exposing the acetaldehyde to the second catalyst system is done in the presence of at least a portion of the H₂ from the reaction product.

2. The method of claim 1, wherein:
(i) the acetaldehyde from the reaction product is exposed to the second catalyst system in a second reactor operated at a temperature ranging from 250° C. to 350° C. and a pressure ranging from 1 psig to 100 psig;
(ii) the ethanol-containing feedstock is exposed to the first catalyst system in a first reactor operated at a temperature ranging from 300° C. to 450° C. and a pressure ranging from 1 psig to 200 psig;
(iii) the acetaldehyde from the reaction product is exposed to the second catalyst system in a second reactor operated at a temperature ranging from 275° C. to 325° C. and a pressure ranging from 1 psig to 100 psig; or
(iv) any combination of two or more of (i), (ii), or (iii).

3. The method of claim 1, further comprising isolating the H₂ from the reaction product.

4. The method of claim 3, wherein a portion of the H₂ from the reaction product is recycled and used in combination with the first catalyst system and/or the second catalyst system.

5. The method of claim 1, further comprising isolating the H₂ from the 1,3-butadiene-containing composition.

6. The method of claim 5, wherein a portion of the H₂ from the 1,3-butadiene-containing composition is used in combination with the first catalyst system and/or the second catalyst system.

7. The method of claim 1, wherein the 1,3-butadiene-containing composition comprises unreacted acetaldehyde.

8. The method of claim 7, wherein the unreacted acetaldehyde is recycled and combined with the acetaldehyde of the reaction product.

9. The method of claim 8, wherein combined unreacted acetaldehyde and acetaldehyde of the reaction product are combined with the second catalyst system.

10. The method of claim 8, further comprising combining ethanol with the unreacted acetaldehyde and/or the acetaldehyde of the reaction product, wherein the ethanol is added in an amount that provides a ratio of ethanol:total acetaldehyde ranging from 1:1 to 3:1.

11. The method of claim 10, wherein combined unreacted acetaldehyde, acetaldehyde of the reaction product, and ethanol are combined with the second catalyst system.

12. The method of claim 1, wherein the first catalyst system comprises copper and an acidic support.

13. The method of claim 12, wherein the acidic support is SBA-16 or SiO₂ and wherein the first catalyst system further comprises a promoter selected from a Group I metal, a Row 4 metal, or a combination thereof.

14. The method of claim 1, wherein the second catalyst system comprises a zirconium oxide, a second catalyst support, and a metal dopant.

15. The method of claim 14, wherein the second catalyst support is a mesoporous SiO₂ material and the metal dopant is silver.

16. The method of claim 14, wherein the second catalyst comprises 0.5 wt % to 8 wt % Ag and 2 wt % to 12 wt % ZrO₂.

17. The method of claim 1, wherein exposing the ethanol-containing feedstock to the first catalyst system is carried out at a temperature ranging from 375° C. to 425° C. and a pressure ranging from 50 psig to 200 psig; and exposing the acetaldehyde of the reaction product to the second catalyst system is carried out at a temperature ranging from 300° C. to 325° C. and a pressure ranging from 35 psig to 50 psig.

18. A method, comprising:
exposing an ethanol-containing feedstock to a first catalyst system in a first reactor to produce a reaction product comprising acetaldehyde and H₂;
passing the acetaldehyde of the reaction product to a second reactor;
passing at least a portion of H₂ of the reaction product to the second reactor; and
exposing the acetaldehyde of the reaction product to a second catalyst system in the second reactor to produce a 1,3-butadiene-containing composition, wherein exposing the acetaldehyde to the second catalyst is done in the presence of the H₂ from the reaction product.

19. The method of claim 18, wherein:
(i) the first catalyst comprises chromium, copper, and a silica support, wherein the copper and chromium are present in amounts that provide a ratio of Cu:Cr ranging from 10:1 to 10:3;
(ii) the second catalyst comprises 0.5 wt % to 8 wt % Ag, 2 wt % to 12 wt % ZrO₂, and mesoporous SiO₂ material;
(iii) the method further comprises recycling a portion of the H₂ from the reaction product to the first reactor;
(iv) the method further comprises recycling any unreacted acetaldehyde from the second reactor by passing the unreacted acetaldehyde back to the second reactor;
(v) the method further comprises adding ethanol to the second reactor;
(vi) the first reactor is operated at a temperature ranging from 350° C. to 425° C. and a pressure of 1 psig to 200 psig; and the second reactor is operated at a temperature of 275° C. to 325° C. and a pressure of 1 psig to 75 psig; or
(vii) any combination of two or more of (i) to (vi).

20. The method of claim 19, wherein the ethanol is added to the second reactor simultaneously with the acetaldehyde from the reaction product and the ethanol and acetaldehyde are introduced to the second reactor at a ratio ranging from 1:1 to 3:1 (ethanol:acetaldehyde).
